(19) United States
(12) Patent Application Publication
(10) Pub. No.: US 2021/0355444 A1
(43) Pub. Date: Nov. 18, 2021

(54) METHOD FOR PRODUCING ERYTHROID CELLS AND/OR ERYTHROCYTES

(71) Applicants: EVER SUPREME BIO TECHNOLOGY CO., LTD, Taichung City (TW); AXEON RESEARCH CORPORATION, Rockville, MD (US)

(72) Inventors: Woei-Cherng SHYU, Taichung City (TW); Henry H. SUN, Rockville, MD (US)

(21) Appl. No.: 17/319,871

(22) Filed: May 13, 2021

Related U.S. Application Data
(60) Provisional application No. 63/024,176, filed on May 13, 2020.

(51) Int. Cl. C12N 5/078

(52) U.S. Cl. CPC C12N 5/0641; C12N 2506/11; C12N 2502/1352; C12N 2501/125; C12N 2501/26; C12N 2501/998; C12N 2500/38; C12N 2501/999; C12N 2501/14; C12N 2501/22; C12N 2501/2303

(57) ABSTRACT

The present disclosure provides a method for producing erythroid cells and/or erythrocytes comprising culturing hematopoietic stem cells (HSCs) or erythroid cells with a population of immortalized mesenchymal stem cells (MSCs) or conditioned medium obtained from the immortalized MSCs, wherein the immortalized MSCs are genetically engineered with a survival gene. Also provided is a method of making a blood product for use in transfusions and a method for increasing hemoglobin synthesis.

### FIELD OF THE INVENTION

The present invention relates to the field of production of erythrocytes. Particularly, engineered stem cells comprising at least a survival gene are used to generate erythroid cells and/or erythrocytes.

### CROSS REFERENCE TO RELATED APPLICATIONS

This application claims priority to U.S. Provisional Application Ser. No. 63/024,176, filed May 13, 2020, which is incorporated by reference herein in its entirety for all purposes.

### BACKGROUND OF THE INVENTION

Though blood transfusion is widely used for various clinical therapies, clinical sources of blood are limited, and the supply of blood for transfusion is dependent on blood donations by volunteers. Progressive reduction of fertility rates has led to a gradual decrease in donor-eligible populations, and a lack of blood source supply is predicted globally (Transfusion 2010; 50:584-588). Moreover, transfusion transmissible diseases remain an important issue. Fortunately, because the culture media for expanding cells may be automatically replaced, it is possible to obtain a large number of target cells beyond the laboratory level.

Discovering technologies for large-scale production of red blood cells (RBC) in vitro is important for producing an alternative source of RBC. Feeder-free incubation in bioreactor system enables manufacturers to develop a xeno-free, cost-effective culture protocol for large-scale in vitro cell generation, which will provide a great advantage for clinical applications (Tissue Engineering. Part C Methods 2011; 17:1131-1137, Biomaterials 2005; 26:7481-7503). However, either the total number of mature RBCs after deleukocyte process or the final RBC enucleation rate was not elucidated. The reproducibility and feasibility of these results should be demonstrated prior to practical utilization.

Therefore, methods to large-scale production of red blood cells are much needed for therapeutic applications.

### SUMMARY OF THE INVENTION

The present disclosure is directed to providing an appropriate microenvironment and stroma such as mesenchymal stem cells (MSCs) to induce erythropoiesis and RBC enucleation.

In one aspect, the present disclosure provides a method for producing erythroid cells and/or erythrocytes comprising culturing hematopoietic stem cells or erythroid cells with a population of immortalized mesenchymal stem cells (MSCs) or a conditioned medium obtained from the immortalized MSCs, wherein the immortalized MSCs are genetically engineered with a survival gene.

In some embodiments, the cell counts of the HSCs or erythroid cells to the cell counts of the immortalized MSCs range from about 100:1 to about 1:100, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 18:1 to about 1:18, from about 16:1 to about 1:16, from about 14:1 to about 1:14, from about 12:1 to about 1:12, from about 10:1 to about 1:10, from about 10:1 to about 1:8, from about 10:1 to about 1:6, from about 10:1 to about 1:4, from about 10:1 to about 1:2, from about 10:1 to about 1:1.

In some embodiments, the HSCs are CD34⁺ HSCs. In another aspect, the HSCs are preferably derived from human umbilical cord blood.

In some embodiments, the survival gene is Akt gene or hepatocyte growth factor (HGF) gene. Preferably, the survival gene is Akt gene.

In some embodiments, the immortalized MSCs are immortalized with human telomerase reverse transcriptase (hTERT).

In one embodiment, the mesenchymal stem cells described herein are umbilical cord mesenchymal stem cells (UMSCs), adipose derived mesenchymal stem cells (ADSCs), or bone marrow mesenchymal stem cells (BMSCs).

In some embodiments, the immortalized MSCs are CD146⁺IGF1R⁻.

In some embodiments, the immortalized MSCs are hypoxia treated.

In one embodiment, the method described herein comprises enhancing HSCs proliferation by culturing the HSCs with the immortalized MSCs or conditioned medium obtained from the immortalized MSCs. In some embodiments, culturing the HSCs with the immortalized MSCs or conditioned medium obtained from the immortalized MSCs for enhancing HSC proliferation is performed for 0.5 to 8 days, such as 0.5 days, 1 day, 1.5 days, 2 days, 2.5 days, 3 days, 3.5 days, 4 days, 4.5 days, 5 days, 5.5 days, 6 days, 6.5 days, 7 days, 7.5 days, or 8 days; preferably for 2 days to 6 days, such as 2 days, 2.5 days, 3 days, 3.5 days, 4 days, 4.5 days, 5 days, 5.5 days, or 6 days; more preferably for 3 days to 5 days, such as 3 days, 3.5 days, 4 days, 4.5 days, or 5 days.

In some embodiments, the method further comprises culturing the HSCs with at least one of stem cell factor (SCF), fms like tyrosine kinase 3 (Flt-3), interleukin 3 (IL-3), vitamin C, and dexamethasone.

In one embodiment, the method described herein comprises inducing the HSCs to differentiate into the erythroid cells by culturing the HSCs with the immortalized MSCs or conditioned medium obtained from the immortalized MSCs. In some embodiments, culturing the HSCs with the immortalized MSCs or conditioned medium obtained from the immortalized MSCs for inducing the HSCs to differentiate into the erythroid cells is performed for 5 days to 20 days, such as 5 days, 6 days, 7 days, 8 days, 9 days, 10 days, 11 days, 12 days, 13 days, 14 days, 15 days, 16 days, 17 days, 18 days, 19 days, or 20 days; preferably for 8 days to 16 days, such as 8 days, 9 days, 10 days, 11 days, 12 days, 13 days, 14 days, 15 days, or 16 days; more preferably for 10 days to 15 days, such as 10 days, 11 days, 12 days, 13 days, 14 days, or 15 days.

In some embodiments, the method further comprises culturing the HSCs with at least one of SCF, erythropoietin (EPO), granulocyte-macrophage colony-stimulating factor (GM-CSF), Flt-3, dexamethasone, IL-3, vitamin C, and platelet rich plasma (PRP).

In one embodiment, the method described herein comprises promoting differentiation and maturation of the erythroid cells by culturing the erythroid cells with the immortalized MSCs or conditioned medium obtained from the immortalized MSCs. In some embodiments, culturing the erythroid cells with the immortalized MSCs or conditioned medium obtained from the immortalized MSCs for promoting differentiation and maturation of the erythroid cells is performed for 0.5 to 8 days, such as 0.5 days, 1 day, 1.5 days, 2 days, 2.5 days, 3 days, 3.5 days, 4 days, 4.5 days, 5 days, 5.5 days, 6 days, 6.5 days, 7 days, 7.5 days, or 8 days; preferably for 2 days to 6 days, such as 2 days, 2.5 days, 3 days, 3.5 days, 4 days, 4.5 days, 5 days, 5.5 days, or 6 days; more preferably for 2 days to 5 days, such as 2 days, 3 days, 4 days, or 5 days.

In some embodiments, the method further comprises culturing the erythroid cells with at least one of heparin, transferrin, SCF, EPO, and vitamin C.

In some embodiments, the concentration of SCF in a medium for culturing the HSCs or erythroid cells ranges from about 10 ng/mL to about 1,000 ng/mL; from about 20 ng/mL to about 800 ng/mL; from about 30 ng/mL to about 600 ng/mL; from about 40 ng/mL to about 400 ng/mL; from about 50 ng/mL to about 300 ng/mL; from about 60 ng/mL to about 250 ng/mL; from about 80 ng/mL to about 200 ng/mL; from about 80 ng/mL to about 150 ng/mL. In some embodiments, the concentration of Flt3 in a medium for culturing the HSCs or erythroid cells ranges from about 10 ng/mL to about 1,000 ng/mL; from about 20 ng/mL to about 800 ng/mL; from about 30 ng/mL to about 600 ng/mL; from about 40 ng/mL to about 400 ng/mL; from about 50 ng/mL to about 300 ng/mL; from about 60 ng/mL to about 250 ng/mL; from about 80 ng/mL to about 200 ng/mL; from about 80 ng/mL to about 150 ng/mL. In some embodiments, the concentration of IL-3 in a medium for culturing the HSCs or erythroid cells ranges from about 1 ng/mL to about 100 ng/mL; from about 2 ng/mL to about 80 ng/mL; from about 4 ng/mL to about 60 ng/mL; from about 6 ng/mL to about 40 ng/mL; from about 8 ng/mL to about 35 ng/mL; from about 10 ng/mL to about 30 ng/mL; from about 12 ng/mL to about 25 ng/mL; from about 15 ng/mL to about 25 ng/mL. In some embodiments, the concentration of vitamin C in a medium for culturing the HSCs or erythroid cells ranges from about 5 μM to about 200 μM; from about 8 μM to about 150 μM; from about 10 μM to about 120 μM; from about 15 μM to about 100 μM; from about 20 μM to about 80 μM; from about 25 μM to about 60 μM; from about 25 μM to about 40 μM; from about 25 μM to about 35 μM. In some embodiments, the concentration of dexamethasone in a medium for culturing the HSCs or erythroid cells ranges from about 0.1 μM to about 10 μM; from about 0.2 μM to about 8 μM; from about 0.3 μM to about 6 μM; from about 0.4 μM to about 4 μM; from about 0.5 μM to about 3 μM; from about 0.6 μM to about 2 μM; from about 0.8 μM to about 1.5 μM; from about 0.8 μM to about 1.2 μM. In some embodiments, the concentration of EPO in a medium for culturing the HSCs or erythroid cells ranges from about 0.1 IU/mL to about 20 IU/mL; from about 0.2 IU/mL to about 18 IU/mL; from about 0.5 IU/mL to about 16 IU/mL; from about 0.8 IU/mL to about 14 IU/mL; from about 1 IU/mL to about 12 IU/mL; from about 2 IU/mL to about 10 IU/mL; from about 3 IU/mL to about 9 IU/mL; from about 4 IU/mL to about 8 IU/mL. In some embodiments, the concentration of GM-CSF in a medium for culturing the HSCs or erythroid cells ranges from about 1 ng/mL to about 50 ng/mL; from about 2 ng/mL to about 45 ng/mL; from about 4 ng/mL to about 40 ng/mL; from about 6 ng/mL to about 35 ng/mL; from about 8 ng/mL to about 30 ng/mL; from about 10 ng/mL to about 25 ng/mL; from about 12 ng/mL to about 25 ng/mL; from about 13 ng/mL to about 20 ng/mL. In some embodiments, the concentration of PRP in a medium for culturing the HSCs or erythroid cells ranges from about 1% to about 100%; from about 2% to about 80%; from about 3% to about 60%; from about 4% to about 40%; from about 5% to about 35%; from about 6% to about 30%; from about 7% to about 20%; from about 8% to about 15%. In some embodiments, the concentration of heparin in a medium for culturing the HSCs or erythroid cells ranges from about 0.1 U/mL to about 20 U/mL; from about 0.2 U/mL to about 18 U/mL; from about 0.5 U/mL to about 16 U/mL; from about 0.8 U/mL to about 14 U/mL; from about 1 U/mL to about 12 U/mL; from about 2 U/mL to about 10 U/mL; from about 3 U/mL to about 9 U/mL; from about 4 U/mL to about 8 U/mL. In some embodiments, the concentration of transferrin in a medium for culturing the HSCs or erythroid cells ranges from about 10 μg/mL to about 2,000 μg/mL; from about 50 μg/mL to about 1,800 μg/mL; from about 100 μg/mL to about 1,600 μg/mL; from about 200 μg/mL to about 1,400 μg/mL; from about 300 μg/mL to about 1,300 μg/mL; from about 40 μg/mL to about 1,200 μg/mL; from about 500 μg/mL to about 1,000 μg/mL; from about 600 μg/mL to about 900 μg/mL.

In one embodiment, the method described herein comprises enhancing HSC proliferation by culturing the HSCs with the immortalized MSCs or conditioned medium obtained from the immortalized MSCs; inducing the HSCs to differentiate into the erythroid cells comprising culturing the HSCs with the immortalized MSCs or conditioned medium obtained from the immortalized MSCs; and promoting differentiation and maturation of the erythroid cells by culturing the erythroid cells with the immortalized MSCs or conditioned medium obtained from the immortalized MSCs.

In one aspect, the present disclosure provides a method of making a blood product for use in transfusions comprising producing erythroid cells and/or erythrocytes by using the method as described herein.

In one aspect, the present disclosure provides a method for increasing hemoglobin synthesis comprising producing erythroid cells and/or erythrocyte by using a method as described herein.

In some embodiments, the hemoglobin is adult hemoglobin.

### BRIEF DESCRIPTION OF THE DRAWINGS

FIG. 1A shows the results of differentiation of adipocyte, chondrocyte, and osteocyte for hTERT-ADSC-Akt and hTERT-ADSC.

FIG. 1B shows the plasmid construction for transduction of AKT and results of western blotting, ELISA, and flow cytometry analysis of hTERT-ADSC-Akt and hTERT-ADSC.

FIG. 1C shows the results of VEGF secretion of hTERT-ADSC-Akt, hTERT-ADSC, hTERT-ADSC-Akt pretreated with hypoxia (H), hTERT-ADSC pretreated with hypoxia (H) at hour 24, 48, and 72 by ELISA.

FIG. 1D shows the results of cell proliferation of CD34⁺ cells cultured with or without conditioned medium on day 5 to day 21.

FIG. 2A shows the results of industrial-scale ex vivo generation of erythropoiesis from CB CD34⁺ cells.

FIG. 2B shows the results of cell proliferation and differentiation to erythroid lineage from stem cells by flow cytometry analysis.

FIG. 2C shows the results of cell proliferation and differentiation to erythroid lineage from stem cells by Wright-Giemsa cell staining.

FIG. 2D shows the results of cell staining by Wright-Giemsa stain on day 1 to day 21.

FIG. 3A shows the results of hemoglobin level of differentiated cells from days 18 to 21.

FIG. 3B shows the photographs of differentiated cells from days 18 to 21.

FIG. 3C shows the results of cell viability.

FIG. 3D shows the results of enucleated RBC rate (CD235a⁺/NucRed⁻) by flow cytometry.

FIG. 4A shows the results of examining hemoglobin subtypes by flow cytometry and hemoglobin expression of cultured erythroid cells and PB.

FIG. 4B shows the results of erythroid markers and hemoglobin content of cultured RBCs.

FIG. 5 shows the results of the percentage of CFSE⁺ cRBC and the view under the confocal microscopy when injecting CFSE-labeled adult peripheral blood RBC (pRBC) or cRBC into CL2MDP-liposome-treated NOD/SCID or nude mice.

### DETAILED DESCRIPTION OF THE INVENTION

Unless defined otherwise, all scientific or technical terms used herein have the same meaning as those understood by persons of ordinary skill in the art to which the present invention belongs. Any method and material similar or equivalent to those described herein can be understood and used by those of ordinary skill in the art to practice the present invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims of the present invention are approximate and can vary depending upon the desired properties sought by the present invention.

The term “a/an” should mean one or more than one of the objects described in the present invention. The term “and/or” means either one or both of the alternatives. The term “a cell” or “the cell” may include a plurality of cells.

As used herein, “erythroid cells” contain nuclei until the cell expels its nucleus and enters the circulation as an anucleate red blood cell (erythrocyte).

The term “ex vivo” generally means outside of a living organism, such as an experiment taking place at an artificial environment created outside of the organism. The term “in vitro” generally describes procedures, tests, and experiments that are performed outside of a living organism.

The term “immortalizing” as used herein refers to inducing, promoting, or enabling cell viability, cell survival, and/or cell proliferation.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

As used herein, the term “derived from” shall be taken to indicate that a particular sample or group of samples has originated from the species specified, but has not necessarily been obtained directly from the specified source.

In the context of cell ontogeny, the adjective “differentiated” or “differentiating” is a relative term. A “differentiated cell” is a cell that has progressed further down the developmental pathway than the cell it is being compared with. Thus, stem cells can differentiate to lineage-restricted precursor cells (such as an HSC), which in turn can differentiate into other types of precursor cells further down the pathway (such as erythroid cells), and then to an end-stage differentiated cell, which plays a characteristic role in a certain tissue type, and may or may not retain the capacity to proliferate further.

The term “genetically engineered” or “genetic engineering” of cells means manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

The term “transduction” or “transduce” means using a virus to deliver the genetic material into cells, wherein the virus can be an integrating or non-integrating virus. The integrating virus used in the present invention can be lentivirus or retrovirus. The integrating virus allows integration of its encoding genes into the transduced cells that are infected with the viral particles. The non-integrating virus can be adenovirus or Sendai virus. Non-viral methods may also be used in the present disclosure such as by transfecting DNA or RNA materials into cells. The DNA materials can be in the form of PiggyBac, minicircle vectors, or episomal plasmids. The RNA material may be in the form of mRNA or miRNA.

The term “expression vector” means the agent carrying foreign genes into cells for expression without degradation. The expression vector in the present invention can be plasmid, viral vectors, and artificial chromosomes.

To induce erythropoiesis and RBC enucleation, it is important to prepare an appropriate microenvironment. Recently, massive expansion of RBCs by CB derived CD34⁺ cells co-cultured on xenogenic (murine) stromal cells (Nat Biotechnol. 2005; 23:69-74). However, for human application, animal derived cells replaced with human stromal cells should be established. Significant increase expansion yield of CD34⁺ cells and enucleation rate of erythroblasts were observed in hTERT stroma co-culture system compared to that in liquid culture without feeder cells (Nat Biotechnol. 2006; 24:1255-6).

The present disclosure uses a survival gene-modified immortalized MSCs to optimize culturing strategies to develop a sequential three-phase co-culture system for ex vivo large-scale generation of human erythrocytes from CB CD34⁺ cells. Accordingly, the present disclosure provides a method for producing erythroid cells and/or erythrocytes comprising culturing hematopoietic stem cells or erythroid cells with a population of immortalized mesenchymal stem cells (MSCs) or a conditioned medium obtained from the immortalized MSCs, wherein the immortalized MSCs are genetically engineered with a survival gene.

The mesenchymal stem cells used in the disclosure can be obtained from different sources, preferably from umbilical cord, adipose tissue or bone marrow. According to different sources, the mesenchymal stem cells are umbilical cord mesenchymal stem cells (UMSCs), adipose derived mesenchymal stem cells (ADSCs), and bone marrow mesenchymal stem cells (BMSCs). In some embodiments of this disclosure, MSCs are isolated and purified from the umbilical cord, and referred to as “umbilical MSC” or “UMSC.” In some embodiments, it is established that the UMSC in this disclosure expresses the same selection of surface markers as the MSC isolated from other bodies, and demonstrates comparable activities.

The immortalized MSCs according to the disclosure are modified to express Akt or HGF. As used herein, the term “modified to express” in the present disclosure refers to transferring an exogenous gene or gene fragment into the mesenchymal stem cells so that they can express the exogenous gene or gene fragment. Preferably, this modification does not alter the differentiation potential of the immortalized MSCs. In another aspect, this modification is preferably be a stable modification, and the expression may be persistent or inducible. The immortalized MSCs according to the disclosure are modified to express Akt or HGF and still have pluripotent differentiation potential, such as, but not limited to, adipogenesis, chondrogenesis, osteogenesis and vascularization, that is similar with the common immortalized MSCs or normal MSCs without Akt or HGF transductions.

Protein kinase B (PKB), also known as Akt, is a serine/threonine-specific protein kinase that plays a key role in multiple cellular processes such as glucose metabolism, apoptosis, cell proliferation, transcription and cell migration. Akt regulates cellular survival and metabolism by binding and regulating many downstream effectors, e.g., Nuclear Factor-κB, Bcl-2 family proteins, master lysosomal regulator TFEB and murine double minute 2 (MDM2). Akt can promote growth factor-mediated cell survival both directly and indirectly. It has been found that hypoxic pre-conditioning of transplanted cells, a brief incubation of cells before transplantation, protects human brain endothelium from ischemic apoptosis through activation of Akt-dependent pathways (Am J Transl Res. 2017; 9: 664-673).

Hepatocyte growth factor (HGF) or scatter factor (SF) is a paracrine cellular growth, motility and morphogenic factor. It is secreted by mesenchymal cells and targets and acts primarily upon epithelial cells and endothelial cells, but also acts on haemopoietic progenitor cells and T cells. Hepatocyte growth factor regulates cell growth, cell motility, and morphogenesis by activating a tyrosine kinase signaling cascade after binding to the proto-oncogenic c-Met receptor. Hepatocyte growth factor is secreted by mesenchymal cells and acts as a multi-functional cytokine on cells of mainly epithelial origin.

The manner of modifying the immortalized MSCs with Akt or HGF is not limited. Preferably, the Akt or HGF is transduced with a transposon or lentivirus; more preferably, the transposon is piggyBac transposon. The results showed that piggyBac transposon can efficiently and stably transfect the MSCs, and the gene modification of piggyBac does not alter the DNA copy number or arrangement of the MSCs.

In some embodiments, an immortalized stem cell utilized in any method described herein comprises an agent that induces cell immortality.

In some embodiments, an immortalized cell is generated by treating the cell with an immortalizing agent. In some embodiments, the immortalizing agent comprises a transgene that expresses or over-expresses a polypeptide that induces cell immortality. In some embodiments, the immortalizing agent comprises a polypeptide that induces cell immortality. In some embodiments, a polypeptide that induces cell immortality is an onco-peptide. Onco-peptides are of any suitable class that induces cell immortality. For example, in certain embodiments, suitable onco-peptides that induce cell immortality are: growth factors and/or mitogens (e.g., PDGF-derived growth factors such as c-Sis); receptor tyrosine kinases, particularly constitutively active receptor tyrosine kinases (e.g., epidermal growth factor receptor (EGFR), thrombocyte-derived growth factor receptor (PDGFR), vascular endothelial growth factor receptor (VEGFR), and HER2/neu); cytoplasmic tyrosine kinases (e.g., Src-family, Syk-ZAP-70 family, and BTK family of tyrosine kinases); cytoplasmic serine/threonine kinases and their regulatory subunits (e.g., Raf kinases, cyclin-dependent kinases, members of the Akt family); regulatory GTPases (e.g., Ras protein); transcription factors (e.g., Myc and HIF-1a); telomerase reverse transcriptases (e.g., TERT or hTERT); and/or factors that activate other onco-peptides (e.g. cyclins, including cyclins A, B, D, and/or E, such as cyclin D1 and D3). In certain embodiments, an onco-peptide is Myc, HIF-1a, Notch-1, Akt, hTERT, or a cyclin. In some embodiments, an onco-peptide is a functional fragment, homolog, or analogue of any onco-peptide that induces cell viability, cell survival and/or cell proliferation, e.g., a functional fragment, homologue, or analogue of Myc, HIF-1a, Notch-1, Akt, hTERT, or a cyclin; preferably, hTERT.

The immortalized MSCs of the present disclosure comprise an expression vector comprising an Akt or HGF gene. In addition to the sequences of Akt or HGF, the vector of the present disclosure comprises one or more control sequences to regulate the expression of the polynucleotide of the present disclosure. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include, among others, promoters, leader sequences, polyadenylation sequences, propeptide sequences, signal peptide sequences, and transcription terminators. In some embodiments, suitable promoters are selected based on host cell selection.

A recombinant expression vector of the present disclosure is disclosed along with one or more expression regulating regions such as a promoter and a terminator, a replication origin, etc., depending on the type of hosts into which they are to be introduced. Non-limiting examples of constitutive promoters include SFFV, CMV, PKG, MDNU3, SV40, Ef1a, UBC, and CAGG.

Various nucleic acid and control sequences described herein are joined together to produce recombinant expression vectors which include one or more convenient restriction sites to allow for insertion or substitution of the polynucleotide of the present disclosure at such sites. Alternatively, in some embodiments, the polynucleotide of the present disclosure is expressed by inserting the polynucleotide or a nucleic acid construct comprising the sequence into an appropriate vector for expression. In some embodiments involving the creation of the expression vector, the coding sequence is located in the vector so that the coding sequence is operably linked with the appropriate control sequences for expression. The recombinant expression vector may be any suitable vector (e.g., a plasmid or virus) that can be conveniently subjected to recombinant DNA procedures and bring about the expression of the polynucleotide of the present disclosure. The choice of vector typically depends on the compatibility of the vector with the host cell into which the vector is to be introduced. The vector may be a linear or closed circular plasmid. In one embodiment, the vector is a viral vector. Examples of viral vectors include retroviral vectors, lentiviral vectors, adenovirus vectors, adeno-associated virus vectors, alphavirus vectors and the like. In a certain embodiment, the viral vector is a lentiviral vector. Lentiviral vectors are based on or derived from oncoretroviruses (the sub-group of retroviruses containing MLV), and lentiviruses (the sub-group of retroviruses containing HIV). Examples of such include, without limitation, human immunodeficiency virus (HIV), equine infectious anaemia virus (EIAV), simian immunodeficiency virus (SIV) and feline immunodeficiency virus (FIV). Alternatively, it is contemplated that other retroviruses can be used as a basis for a vector backbone such as murine leukemia virus (MLV).

In some embodiments, the immortalized MSCs of the present disclosure have been tested in various differentiation assays to establish their comparability to the conventional MSC isolated from other locations of the mammalian body. The differentiation assays include adipogenic differentiation, osteogenic differentiation, and chondrogenic differentiation. In some embodiments, the differentiation assay further includes neuronal cell differentiation.

In some embodiments of the disclosure, Akt-modified hTERT-MSCs are applied to optimize culturing strategies to develop a sequential three-phase co-culture system with hTERT-MSC-Akt for ex vivo large-scale generation of human erythrocytes from CB CD34⁺ cells. To induce erythropoiesis and RBC enucleation, it is important to prepare an appropriate microenvironment with adequate cytokine supplements and stroma such as mesenchymal stem cells (MSCs).

Preferably, the immortalized MSCs as described in the disclosure are hypoxia treated. In one embodiment of the disclosure, hypoxia pretreatment of the immortalized MSCs modified with Akt induced more VEGF secretion in the conditioned medium than that in immortalized MSCs without Akt.

In one embodiment of the disclosure, ex vivo expansion of erythroid cells through a combined liquid culture with MSCs co-culturing systems or derived conditioned medium starting from cord blood derived CD34⁺ HSCs were incubated for more than 25 days in erythroid proliferation and differentiation conditions, which resulted in a more than 10⁶-10⁷-fold expansion within 25 days under optimal conditions. Homogeneous erythroid cells were characterized by cell morphology, and flow cytometry. Furthermore, terminal erythroid maturation was improved by adding conditioned medium or co-culturing with CD146⁺IGF1R⁺ immortalized MSCs carrying Akt (hTERT-ADSC-Akt). Cultured erythroid cells underwent multiple maturation events, including decrease in size, increase in glycophorin A (CD235a) expression, and nuclear condensation, which resulted in extrusion of the pycnotic nuclei in as much as 80% of the cells or more. Importantly, they possessed the capacity to express the adult definitive β-globin chain (HbA) upon further maturation. The oxygen equilibrium curves of the cord blood-differentiated red blood cells (RBCs) are comparable to normal RBCs. The large number and purity of erythroid cells and RBCs produced from cord blood make this method useful for providing a basis for future production of available RBCs for transfusion.

In an embodiment, the erythroid cells are from in vitro or ex vivo expanded and differentiated HSCs. In some embodiments, the erythroid cells comprise hematopoietic precursor cells, e.g., CD34⁺ cells.

In an embodiment, the erythroid cells are obtained from blood. The erythroid cells obtained from blood or from in vitro or ex vivo expanded and differentiated HSCs can both be applied for further producing erythrocytes.

In certain embodiments, the immortalized HSCs are continuously maintained successfully as an immortalized ESC line.

In one embodiment, the method described herein comprises a first phase of enhancing HSCs proliferation by culturing the HSCs with the immortalized MSCs or conditioned medium obtained from the immortalized MSCs. In some embodiments, the first phase of the method further comprises culturing the HSCs with at least one of stem cell factor (SCF), fms like tyrosine kinase 3 (Flt-3), interleukin 3 (IL-3), vitamin C, and dexamethasone.

In one embodiment, the method described herein comprises a second phase inducing the HSCs to differentiate into the erythroid cells by culturing the HSCs with the immortalized MSCs or conditioned medium obtained from the immortalized MSCs. In some embodiments, the second phase of the method further comprises culturing the HSCs with at least one of SCF, erythropoietin (EPO), granulocyte-macrophage colony-stimulating factor (GM-CSF), Flt-3, dexamethasone, IL-3, vitamin C, and platelet rich plasma (PRP).

In one embodiment, the method described herein comprises a third phase of promoting differentiation and maturation of the erythroid cells by culturing the erythroid cells with the immortalized MSCs or conditioned medium obtained from the immortalized MSCs. In some embodiments, the third phase of the method further comprises culturing the erythroid cells with at least one of heparin, transferrin, SCF, EPO, and vitamin C.

In one embodiment of the disclosure, ex vivo expansion of erythroid cells through a combined liquid culture with MSCs co-culturing systems or derived conditioned medium starting from cord blood derived CD34⁺ HSCs were incubated for more than 25 days in erythroid proliferation and differentiation conditions, which resulted in a more than 10⁶-10⁷-fold expansion within 25 days under optimal conditions. Homogeneous erythroid cells were characterized by cell morphology, and flow cytometry. Furthermore, terminal erythroid maturation was improved by adding conditioned medium or co-culturing with CD146⁺IGF1R⁺ immortalized MSCs carrying Akt (hTERT-ADSC-Akt). Cultured erythroid cells underwent multiple maturation events, including decrease in size, increase in glycophorin A (CD235a) expression, and nuclear condensation, which resulted in extrusion of the pycnotic nuclei in up to over 80% of the cells. Importantly, they possessed the capacity to express the adult definitive β-globin chain (HbA) upon further maturation. The oxygen equilibrium curves of the cord blood-differentiated red blood cells (RBCs) are comparable to normal RBCs. The large number and purity of erythroid cells and RBCs produced from cord blood make this method useful for providing a basis for future production of available RBCs for transfusion.

In an embodiment, the erythroid cells are from in vitro or ex vivo expanded and differentiated HSCs. In some embodiments, the erythroid cells comprise hematopoietic precursor cells, e.g., CD34⁺ cells.

In an embodiment, the erythroid cells are obtained from blood. The erythroid cells obtained from blood or from in vitro or ex vivo expanded and differentiated HSCs can be both applied for further producing erythrocytes.

In certain embodiments, the immortalized HSCs are continuously maintained successfully becoming an establishing an immortalized ESC line.

The conditioned medium as used herein refers to a medium that is conditioned by culture of the immortalized MSCs. Such a conditioned medium comprises molecules secreted by the immortalized MSCs, including unique gene products. Such a conditioned medium, and combinations of any of the molecules comprised therein, particularly including proteins or polypeptides, may be used in the treatment of disease. They may be used to supplement the activity of, or in place of, the immortalized MSCs, for the purpose of, for example, producing erythroid cells and/or erythrocytes.

In one aspect, the present disclosure provides a method of making a blood product for use in transfusions comprising the method for producing erythroid cells and/or erythrocytes as described herein.

In one aspect, the present disclosure provides a method for increasing hemoglobin synthesis comprising the method for producing erythroid cells and/or erythrocyte as described herein.

It is to be understood that if any prior art publication is referred to herein, such reference does not constitute an admission that the publication forms a part of the common general knowledge in the art.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### EXAMPLES

Methods and Materials:

Separation and Collection of CD34⁺ Cell

Umbilical cord blood (CB) samples (O-type) from normal full-term deliveries were provided by healthy adult volunteers after obtaining written informed consent approved by the China Medical University Institutional Review Board (Taichung, Taiwan). To obtain CB CD34⁺ cells, we isolated low-density mononuclear cells from CB by ficoll-hypaque (SIGMA®) centrifugation, and then purified CB CD34⁺ cells from mononuclear cells via super magnetic microbead selection using Mini-MACS columns (MILTENYI®). The purity of isolated CD34⁺ cells ranged from 90% to 99%, as determined by flow cytometry using anti-human CD34 mAb conjugated with phycoerythrin (PE) (BD®).

Preparation, Isolation, and Characterization of Primary UMSCs

The collected human umbilical cord tissues approved by the Institutional Review Board (IRB) of the China Medical University Hospital, Taichung were washed three times with Ca²⁺ and Mg²⁺-free PBS (DPBS, LIFE TECHNOLOGY®). They were mechanically cut by scissors in a midline direction and the vessels of the umbilical artery, vein and outlining membrane were dissociated from the Wharton's jelly (WJ). The jelly content was then extensively cut into pieces smaller than 0.5 cm³, treated with collagenase type 1 (SIGMA®, St Louis, USA) and incubated for 3 h at 37° C. in a 95% air/5% CO₂ humidified atmosphere. The explants then were cultured in DMEM containing 10% fetal calf serum (FCS) and antibiotics at 37° C. in a 95% air/5% CO₂ humidified atmosphere. They were left undisturbed for 5-7 days to allow for migration of the cells from the explants. The cellular morphology of umbilical cord-derived mesenchymal stem cells (UMSCs) became homogenously spindle shaped in cultures after 4-8 passages, and the specific surface molecules of cells from the WJ were characterized by flow cytometric analysis. The cells were detached with 2 mM EDTA in PBS, washed with PBS containing 2% BSA and 0.1% sodium azide (SIGMA®) and incubated with the respective antibody conjugated with fluorescein isothiocyanate (FITC) or phycoerythrin (PE) including CD13, CD29, CD44, CD73, CD90, CD105, CD166, CD49b, CD1q, CD3, CD10, CD14, CD31, CD34, CD45, CD49d, CD56, CD117, HLA-ABC, and HLA-DR (BD®, PHARMINGEN®). Thereafter, the cells were analyzed using a Becton Dickinson flow cytometer (BD®).

Plasmid Construction

Akt cDNA from plasmids of Akt (0.1 μg) (pCMV6-myc-DDK-Akt, ORIGENE®) were transferred into pIRES (CLONTECH®) or pSF-CMV-CMV-SbfI (OXFORD GENETICS®) by specific restriction enzyme linker (EcoR1, Nhe1, BamH1 and Not1) to build as the construct of pSF-Akt-GFP.

Construction of the piggyBac Transposon System for Stable Cell Lines

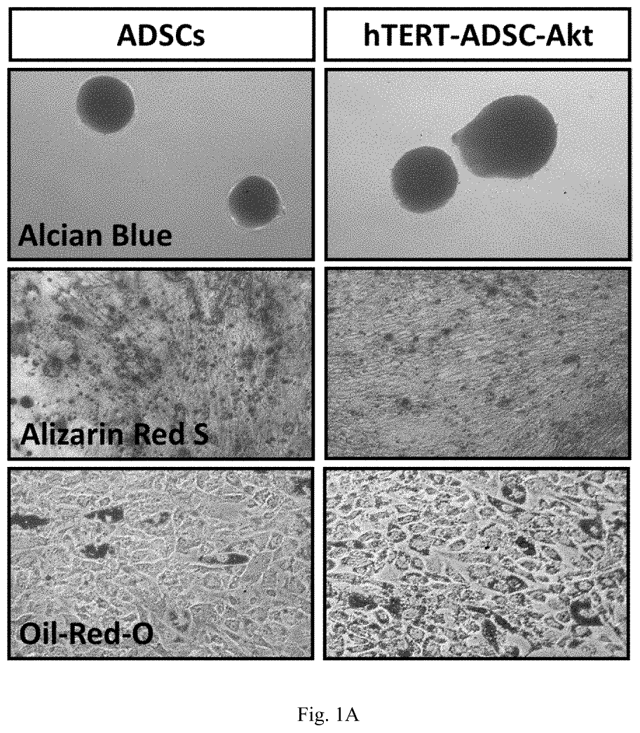

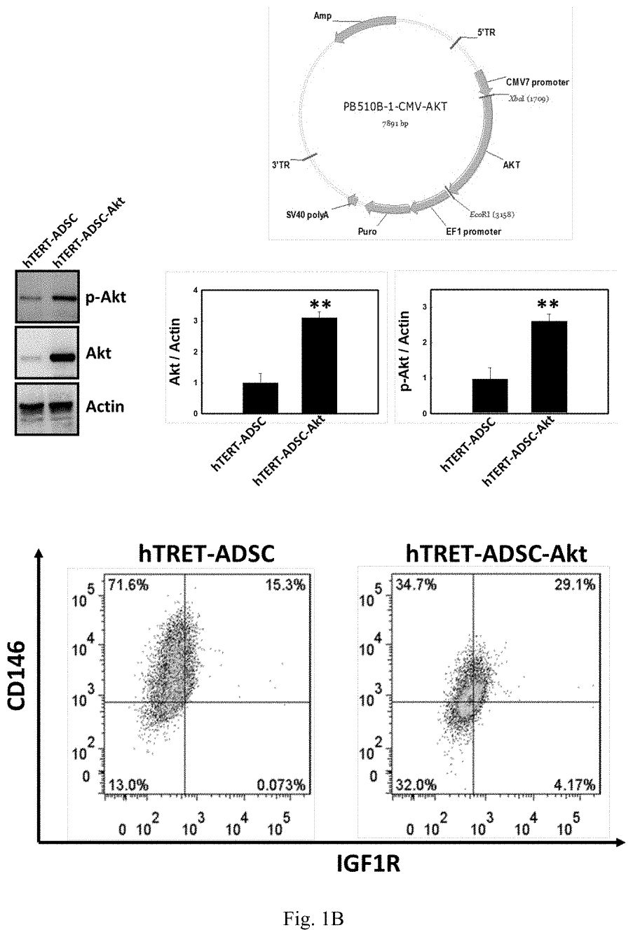

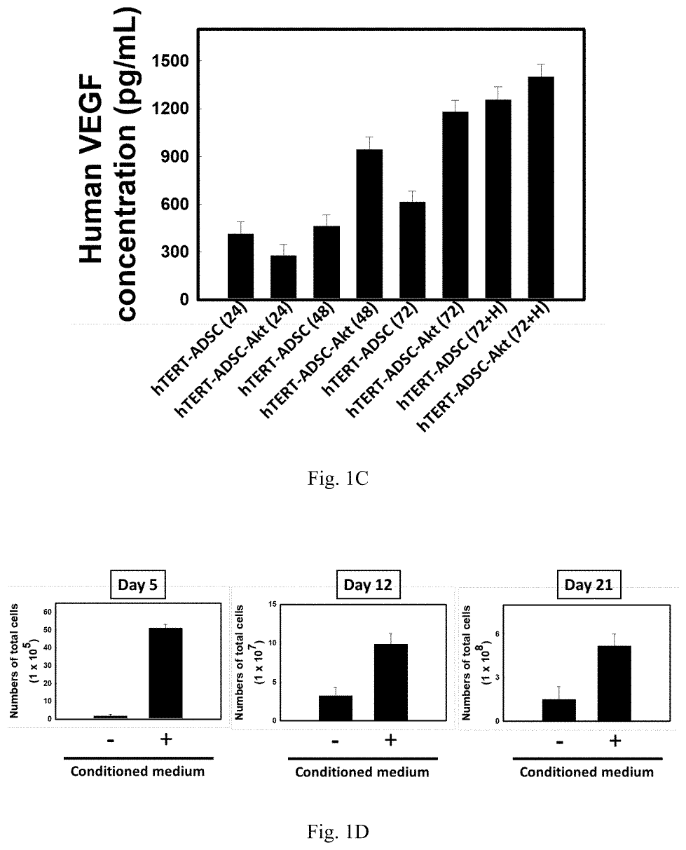

A piggyBac vector pPB-CMV-MCS-EF1α-RedPuro, which contains the multiple cloning sites (MCS), piggyBac terminal repeats (PB-TRs), core insulators (CIs) and puromycin selection maker (BSD) fused with RFP driven by the human EF1α, was used as the base vector (SYSTEM BIOSCIENCES®). DNA fragment containing Akt (from pSF-Akt) was PCR amplified and subcloned into the pPB-CMV-MCS-EF1α-RedPuro vector, in front of the coding region of EF1α. Detailed information regarding vector constructions (pPB-Akt) is shown in FIG. 1B. To generate hTERT-ADSC-Akt stable cells, the above pPB-Akt plasmids were co-transfected with a piggyBac transposase expression vector (SYSTEM BIOSCIENCES®) into hTERT-ADSCs (SCRC-4000™, ATCC) by electroporation (AMAXA NUCLEOFECTOR II®, Lonza). Stably transfected cells were selected in the presence of puromycin.

Total Protein Extraction, Western Blotting, and ELISA

Cells were lysed in a buffer containing 320 mM sucrose, 5 mM HEPES, 1 μg/mL leupeptin, and 1 μg/mL aprotinin. Lysates were centrifuged at 13,000 g for 15 min. The resulting pellet was resuspended in sample buffer (62.5 mM Tris-HCl, 10% glycerol, 2% SDS, 0.1% bromophenol blue, and 50 mM DTT) and subjected to SDS-polyacrylamide gel (4-12%) electrophoresis. The gel was then transferred to a Hybond-P nylon membrane. This was followed by incubation with appropriately diluted antibodies to Akt (1:200, NOVUS BIOLOGICALS®). Membrane blocking, primary and secondary antibody incubations, and chemiluminescence reactions were conducted for each antibody individually according to the manufacturer's protocol. The intensity of each band was measured using a Kodak® Digital Science 1D Image Analysis System (EASTMAN KODAK®). In addition, the total amount of VEGF, HGF (Quantikine ELISA kit, R&D®) in the medium was measured according to the manufacturer's instructions. Optical density was measured using a spectrophotometer (MOLECULAR DEVICES®), and standard curves were generated with the program SOFTmax (MOLECULAR DEVICES®).

In Vitro Differentiation Assays

For adipocyte differentiation, cells were cultured in medium containing low-glucose DMEM, 1×ITS (SIGMA®), 1 mg/ml LA-BSA (SIGMA®), 1 mM hydrocortisone (SIGMA), 60 mM indomethacin (SIGMA®), 0.5 mM isobutylmethylxanthine (SIGMA®) and 10% horse serum (INVITROGEN®). To assess adipogenic differentiation, cells were stained for 10 min at room temperature with 0.3% oil red O (SIGMA®) as an indicator for intracellular lipid accumulation and were counterstained with hematoxylin. For chondrocyte differentiation, cells were cultured in medium containing 90% high-glucose DMEM, 10% FBS, 1×ITS, 1 mg/ml LABSA, 50 nM dexamethasone and 60 μM transforming growth factor-01 (TGF-b1) (R&D SYSTEMS®). Alcian Blue/Sirius red staining (SIGMA®) was carried out by applying 0.5% Alcian Blue 8GX for proteoglycan-rich cartilage matrix and 1% Sirius red F3B for collagenous matrix. Osteogenic differentiation was conducted in confluent monolayer cultures of APSCs grown in high-glucose DMEM containing 10% FCS, 100 U/ml penicillin, 100 mg/ml streptomycin, 50 mg/ml L-ascorbic acid 2-phosphate, 10 mM b-glycerophosphate, and 100 nM dexamethasone. Osteogenesis was determined using alizarin red S staining (1%) to detect calcium mineralization.

Preparation of MSC-Derived Conditioned Medium

CD146⁺IGF1R⁺ hTERT-ADSC-Akt (1×10⁶) were allowed to grow until 80-90% confluency in culture flasks. The cells were then conditioned with 10 mL serum-free CellGenix SCGM (CELLGENIX®). The conditioned medium was collected after 24 h and sterilized with 0.2 mm syringe filter (THERMO FISHER®). The prepared conditioned media were kept at −80° C. until use.

Hypoxia Procedure

Cells cultured at 37° C. in 5% CO₂-humidified incubators were treated in normoxic (21% O₂) or various hypoxic conditions (1%, 3% and 5% O₂) at different time points (24 hr, 48 hr, or 72 hr). Hypoxic cultures were cultivated in a two-gas incubator (JOUAN INC, Winchester, Va.) equipped with an O₂ probe to regulate N₂ gas levels. Cell number and viability were evaluated using trypan blue exclusion assay.

Cytokine Array

Whole proteins were extracted using lysis buffer supplemented with a protease and phosphatase inhibitor cocktail (INVITAGEN). Using the Human cytokine array panel (R&D SYSTEMS®), 100 mg exosomal protein was tested for cytokine levels under the manufacturer's instructions. Briefly, exosome lysates were mixed with the detection antibody cocktail and incubated with the membrane that contains 40 different anti-cytokine capture antibodies overnight at 4° C. After incubation with Streptavidin-HRP, the membranes were incubated with chemiluminescent substrate and exposed to X-ray film. The pixel densities of proteins were quantified using ImageJ 1.47 software.

Collection and Isolation of CD34⁺ Cell from Cord Blood (CB)

Umbilical CB (CB) samples (O-type) were collected in the China Medical University Hospital. The study was approved by the Hospital's Institution Review Board (IRB) of Committee on Ethics. Isolation of CD34⁺ cells from CB was performed by super-magnetic microbead conjugated with anti-CD34 mAb selection using Mini-MACS columns (MILTENYI®). The purity of isolated CD34⁺ cells was determined by flow cytometry (BD®).

Incubation of CB CD34⁺ Cells Cell-Free System or on hTERT-ADSC-Akt (First-Phase)

To expand HSCs from CB CD34⁺ cells in first phase culture (days 1-4), CB CD34⁺ cells (1×10⁵/mL) were seeded in cell-free system with conditioned medium that had been plated in a 75-cm² flask (CORNING®) with 10 mL serum-free SCGM (CELLGENIX®) containing albumin, and insulin supplemented with 100 ng/mL recombinant human stem cell factor (SCF, GIBCO®), 1 μM dexamethasone (Dex, SIGMA®), 30 μM Vitamin C (Vit-C, SIGMA®), and 1 ng/mL recombinant human interleukin-3 (IL-3, GIBCO®) at 37° C. in 5% CO₂. Media was partially replenished every 2 days.

Incubation of HSCs for Erythroid Cells Expansion and Differentiation on hTERT-ADSC-Akt (Second and Third Phases)

On day 8, for erythroblast expansion, the cells (1 to 2×10⁶ cells/mL) were maintained in CellGenix SCGM (CELLGENIX®) with/without hTERT-ADSC-Akt derived conditioned medium and supplemented with 100 ng/mL recombinant human stem cell factor (SCF, GIBCO®), 6 U/mL recombinant human erythropoietin (EPO, SIGMA®), 1 ng/mL IL-3 (GIBCO®), 30 μM Vitamin C (Vit-C, SIGMA®), 5% platelet rich plasma (PRP, AVENTACELL®), 15 ng/mL GM-CSF (GIBCO®), 100 ng/mL Flt3 (GIBCO®) and 1 μM dexamethasone (SIGMA®) for 12-14 days in a 75-cm² flask (CORNING®) or Hyperflask (CORNING®) (second phase). Next, differentiation and enucleation (third phase) of the erythroblasts was seeded on a monolayer of CD146⁺IGF1R⁺ hTERT-ADSC-Akt (1×10⁶) for induction in differentiation medium refreshed (half) containing CellGenix SCGM (CELLGENIX®) supplemented with EPO (10 U/mL), SCF (100 ng/mL), transferrin (700 ug/ml, SIGMA®), 30 μM Vitamin C (Vit-C, SIGMA®) and heparin (5 U/mL, SIGMA®) for 3 days of differentiation. For leukocyte filtration, cultured cells were then purified using a 60 ml deleukocyte filter (Immuguard III-RC, TERUMO®). After filtering, the filter was washed 2 times and resuspended in with 25 mL CellGenix SCGM (CELLGENIX®). Cells were centrifuged at 1600 rpm for 5 min in order to obtain packed RBC. Cells cultured were harvested and stored at 4° C. for 4 weeks in a citrate phosphate dextrose adenine (CPDA-1) preservative-based solution as previously described.

Flow-Cytometry

For the analysis of the cell surface-marker expression, cells were detached with 2 mM EDTA in PBS, washed with PBS containing BSA (2%) and sodium azide (0.1%), and then incubated with the respective antibody conjugated with fluorescein isothiocyanate (FITC) or phycoerythrin (PE) until analysis. As a control, cells were stained with mouse IgG1 isotype-control antibodies. The antibodies to CD34, CD36, CD45, CD71, CD146, IGF1R and CD235a for flow cytometry were purchased from BD Biosciences. Cells were analyzed using a FACScan (BD®) with the CellQuest Analysis (BD BIOSCIENCES®) and FlowJo software v.8.8 (TREESTAR Inc.). Results are expressed by the percentage of positively stained cells relative to total cell number. For quantitative comparison of surface protein expression, the fluorescence intensity of each sample was presented as median fluorescence intensity (MFI). Nuclei were stained with NucRed Live 647 (NucRed, INVITROGEN®). Enucleation rate was calculated from the CD235a⁺/NucRed⁻ portion at days 18-21. Data were analyzed using a FACScan (BD®) with CellQuest Analysis (BD BIOSCIENCES®) and FlowJo v.8.8 (TREESTAR®).

Cell Counts and Morphological Analysis of the Cultured Cells

Cell numbers and morphology were assessed by the automated cell counter Z1 (BECKMAN COULTER®) and Wright-Giemsa staining (SIGMA®), respectively.

Hemoglobin Content Detection and Oxygen Dissociation Curve

The hemoglobin (Hb) content of cultured cells and RBCs from a healthy volunteer was quantified photometrically at 540 nm using Drabkin's reagent (SIGMA®). For measuring hemoglobin status by flowcytometry, cells were fixed, permeabilized, and tagged with fetal hemoglobin-FITC (Hb-F, BD®), hemoglobin beta-PE (Hb-β, Santa Cruz). Oxygen dissociation curves for Hb in RBC were measured using a Hemox-Analyzer (TCS SCIENTIFIC CORP).

Reverse Transcription Quantitative Polymerase Chain Reaction (RT-qPCR)

Cultured RBC were collected and evaluated to determine RNA expression of levels of ε-globin, γ-globin, β-globin, ζ-globin, and α-globin. Total RNA was isolated using the RNeasy mini kit (QIAGEN®), and the Superscript 3 First-strand for RT-PCR Synthesis (LIFE TECHNOLOGIES®) was used to obtain complementary DNA (cDNA). Quantitative PCR assay was performed using gene-specific primers and probes in the Mx3000P (AGILENT TECHNOLOGIES®).

Hemoglobin (Hb) Analysis by HPLC

To determine the proportion of Hb A and F, lysates of erythroblasts, CD34-derived RBC, and CB were measured by high-performance liquid chromatography (HPLC) on a cation-exchange TSK gel G7 HSi column (SIGMA®) photometrically at 610 nm. Analyses were performed on washed cell pellets with use of the Bio-Rad Variant II dual program (BIO-RAD LABORATORIES®) according to the manufacturer's instructions.

In Vivo Mouse Study

Eight-week-old NOD/SCID or NSG mice purchased from National Laboratory Animal Center, Taiwan were used. All animal experiments were performed in accordance with institutional guidelines approved by the Animal Committee of the China Medical University. Before cultured RBC (cRBC) injection, mice were injected intravenously with CL2MDP-liposome (FORMUMAX®) two times (days −3, and day −1) to deplete macrophages. cRBC (1.5×10⁸) or adult peripheral RBC (pRBC) (1.5×10⁸) labeled with CFSE (LIFE TECHNOLOGIES®) were injected into the femoral vein of mice. Heparinized peripheral blood of NOD/SCID mice was aspirated from the retro-orbital vein puncture at 10, 20, 40, 60, 120, 240, 480 and 720 minutes after inoculation, and once daily thereafter up to 3-5 days. Cells were counted and double stained with anti-human CD71, anti-human CD235a and nucleic acid dye of NucRed Live 647 (NUCRED®), and analyzed by flow cytometry. Non-CL2MDP-liposome treated mice (control) were also transfused and analyzed to assess the effects of murine macrophages on the inoculated cells.

### Example 1: Optimization of Culturing Protocol for Expanding Human Erythrocytes from Hematopoietic Stem Cells

A three-phase protocol was developed with regular medium formulas for the ex vivo expansion and differentiation of human erythrocytes from cord blood (CB) CD34⁺ cells.

To isolate the hematopoietic stem cells, CB sample volume collected for CD34⁺ selection was 95±7.8 mL (n=8). The purity and cell count of the isolated CD34⁺ cells were 95.5±2.1 percent and 3.1±0.3×10⁶. The viability of CD34⁺ cells assessed by 7-aminoactinomycin D (7-AAD) was 97.6±0.4 percent.

The ratio of the cell counts of the CD34⁺ cells to the cell counts of the immortalized MSCs (hTERT-ADSC-Akt or hTERT-ADSC) was about 10:1.

To show the advantage and stem cells self-renewal potential of hTERT-ADSC-Akt, mesenchymal differentiation of adipocyte, chondrocyte and osteocyte is the same between hTERT-ADSC and hTERT-ADSC-Akt (FIG. 1A). Significantly increased expression of Akt and p-Akt was noted in the hTERT-ADSC-Akt compared to that in hTERT-ADSC (FIG. 1B). Importantly, enhanced level of stemness surface markers on CD146⁺IGF1R⁺ was present in the hTERT-ADSC-Akt group (FIG. 1B). Consistently, hypoxia pretreatment of hTERT-ADSC-Akt induced more VEGF secretion in the conditioned medium than that in hTERT-ADSC by ELISA (FIG. 1C).

To demonstrate the enhancement of cell proliferation in step 1 (day 1 to day 4) by the conditioned medium, isolated CD34⁺ cells were expanded for 4 days to increase the amount of CD34⁺ hematopoietic stem cells (HSCs). CellGenix SCGM (CELLGENIX®) with hTERT-ADSC-Akt conditioned medium were prepared to supplement with SCF at 100 ng/ml, Flt3 at 100 ng/mL, IL-3 at 20 ng/ml, Vit-C at 30 μM and Dex at 1 μM, which induced higher expansion fold about 30±1.6 than that without conditioned medium (FIG. 1D).

To induce the expanded HSCs to differentiate into the erythroid lineage in step 2 (day 5 to day 18), we optimized combinations and concentrations of growth factors with or without hTERT-ADSC-Akt conditioned medium for generating human erythroid progenitors ex vivo including CellGenix SCGM (CELLGENIX®) supplemented with the SCF at 100 ng/ml, EPO at 6 IU/ml, GM-CSF at 10 ng/mL, Flt3 at 100 ng/mL and dexamethasone at 1 μM and IL-3 at 20 ng/ml for erythroid differentiation (FIG. 1D). Importantly, the addition of 5% human platelet rich plasma (PRP) significantly improved cell yield.

To promote further differentiation and maturation of cultured erythroid cells in step 3 (day 19 to day 21), cultured erythroid cells co-cultured with hTERT-ADSC-Akt were incubated in CellGenix SCGM (CELLGENIX®) supplemented with heparin (5 IU/ml) and transferrin (700 μg/ml), SCF (100 ng/ml) and EPO (10 IU/ml) to achieve a higher level of total erythrocyte cell numbers (FIG. 1D). SCF, EPO, GM-CSF, Flt3 and IL-3 with PRP at 5% demonstrated a significant expansion of cultured erythroid cells.

### Example 2: Scale-Up Expansion of Human Erythrocytes from CD34⁺ Cells

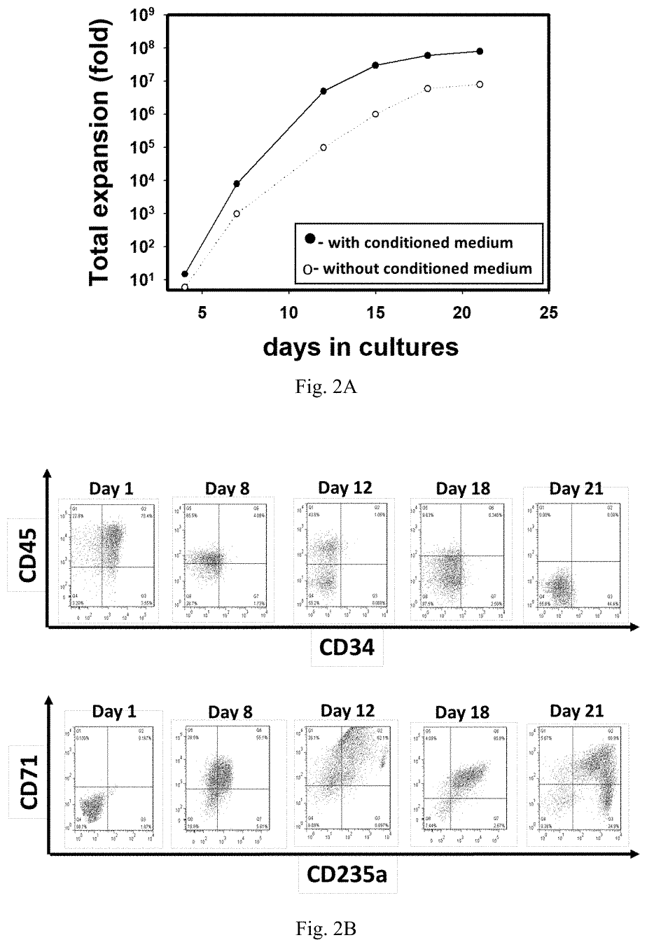

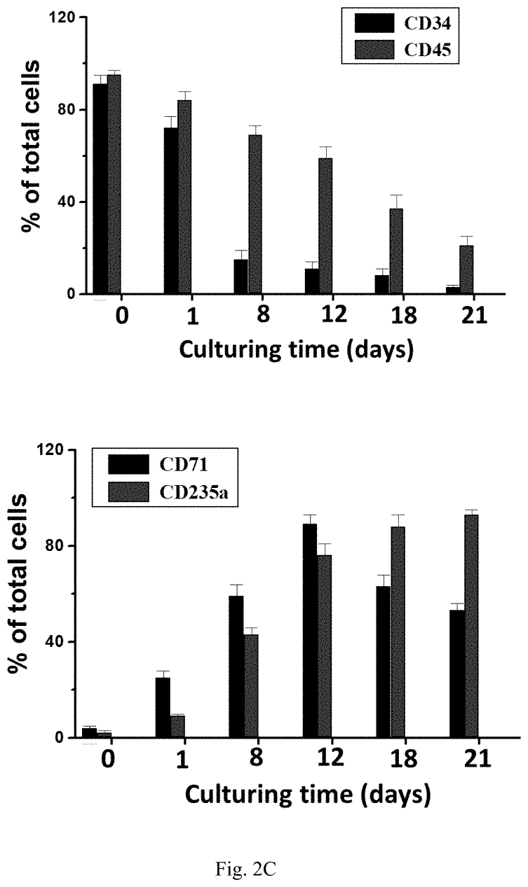

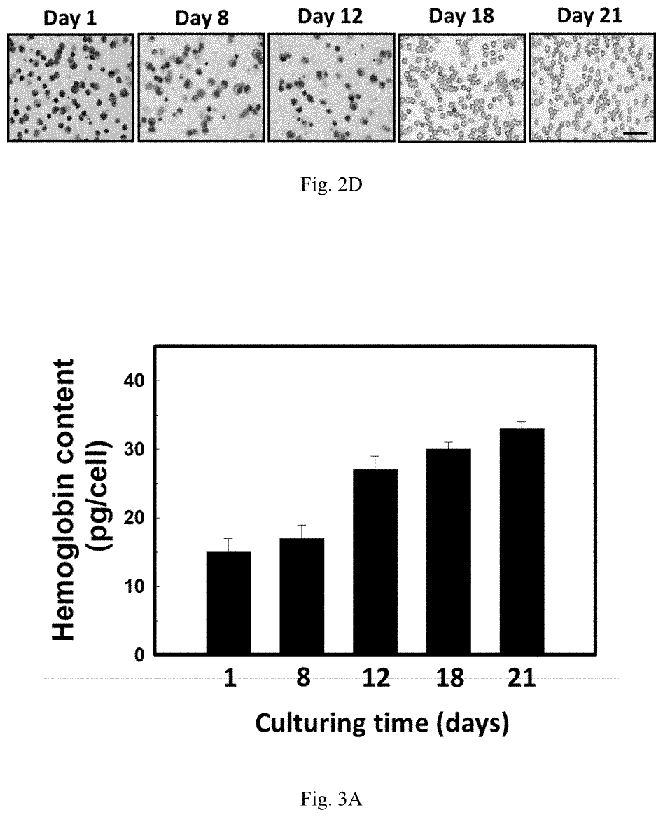

Industrial-scale ex vivo generation of erythropoiesis from CB CD34⁺ cells was performed in the Hyperflask culturing system (CORNING®) with the above-mentioned optimized strategy. 1×10⁵ cells/mL CB CD34⁺ were able to generate 2.9×10¹¹ total red blood cells (RBCs) with a 55.0% enucleation rate by the use of about 100-120 liter medium. The ratio of the cell counts of the CD34⁺ cells to the cell counts of the immortalized MSCs was about 10:1. Ex vivo scale-up fold of total cells expanding slowly during the initial culture period (step 1 from day 1 to day 4) was shown in the growth curve (FIG. 2A). Then, in step 2 from day 5 to day 18, cells maintained a high proliferation rate to an exponential growth phase (FIG. 2A). Cells can be expanded to about 2.9×10⁶-fold and 8.9×10⁷-fold increase by day 12 and day 15, respectively. Finally, in step 3, total cell generation got a slow expansion rate and achieved a plateau of about 2×10⁸-fold (1.4-2.53 10⁸-fold) by day 21-22. More expansion of cell yield revealed in the culturing protocol administrated with hTERT-ADSC-Akt conditioned medium than that without conditioned medium (FIG. 2A). If culture has been maintained, cell growth would decrease in relation to cell differentiation and death observed from day 22-23 (data not shown).

Cell proliferation and differentiation to erythroid lineage from stem cells were morphologically examined by Wright-Giemsa cell staining and flow-cytometric analysis. Initially, as expected, the expression of erythroid markers of CD71 and CD235a was low, whereas a high level of HSCs markers (CD34, and CD45) was expressed by isolated CD34⁺ cells (day 0) (FIGS. 2B-2C). Progressively, the percentage of CD34⁺ decreased significantly to about 1%-2% after 21 days of differentiation (FIGS. 2B-2C). Conversely, the expression of CD235a increased gradually and maintained a high level after cell differentiation (FIG. 2B-2C). In the differentiated cells, the expression of CD71 rapidly increased to peak on day 8, and then continuously down-regulated following the differentiation process (FIG. 2B-2C). Finally, the completely differentiated cells robustly expressed CD235a (90.1%±6.2%) and weakly expressed CD71 (54.0%±7.2%) on day 21 (FIGS. 2B-2C). Cell staining by Wright-Giemsa stain revealed sequentially that cell morphology changed from initial proerythroblast to enucleated RBCs; a pure erythroid phenotype was noted in this population (FIG. 2D).

### Example 3: Enhancement of Erythroid Cell Proliferation and Maturation

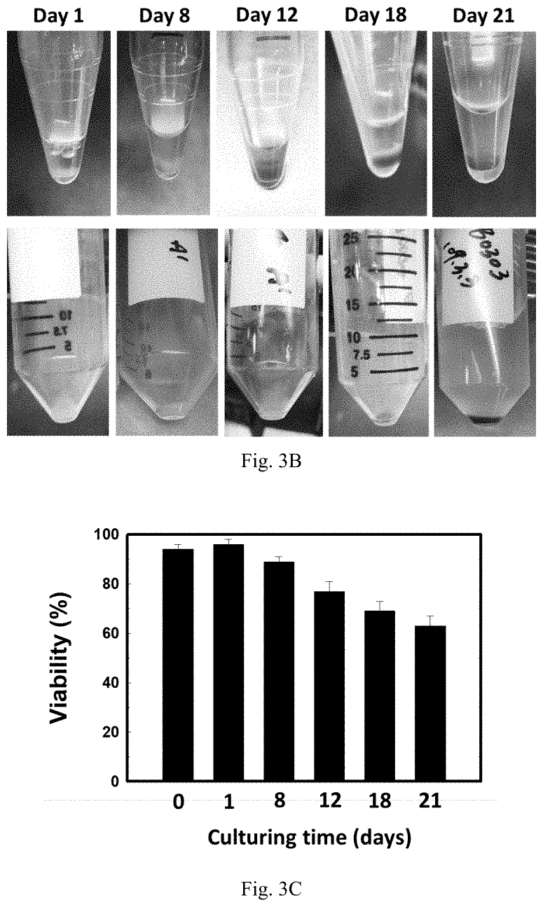

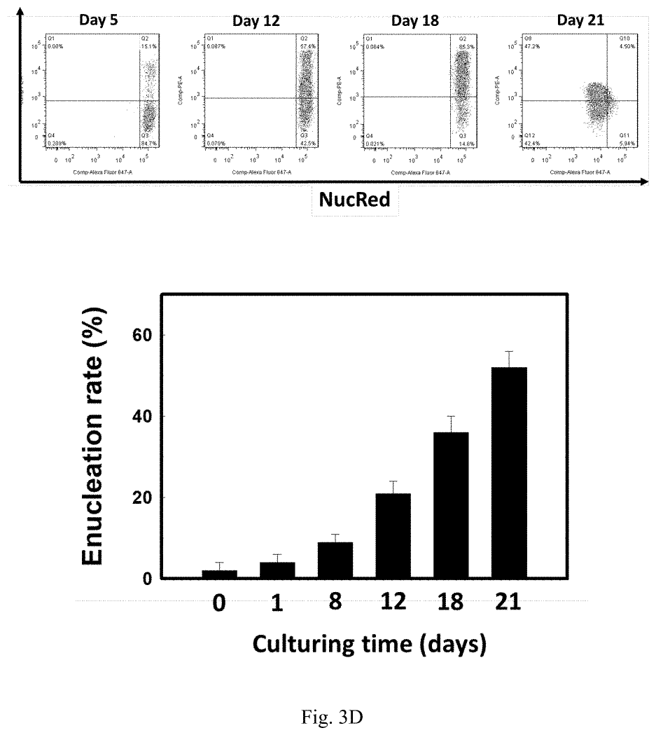

The hemoglobin level of differentiated cells increased gradually (from 17.6±2.2 pg/cell to 30.3±1.8 pg/cell) to reach approximately the content of normal human RBCs (27-33 pg/cell) from day 18 to 21 (FIG. 3A). Moreover, increased hemoglobin synthesis following cell differentiation made the color of the cell pellet change from white-light pink to red after centrifugation (FIG. 3B).

Good cell morphology was noted during the immature stage until day 11, but dead cells were observed from day 18. The cell viability on the final culture day showed intact cell membrane (FIG. 3C). The enucleated RBC rate (CD235a⁺/NucRed⁻) by flowcytometry was significantly increased by erythrocyte co-culturing with hTERT-ADSC-Akt until a mean of 54-65% at day 21 compared to without coculturing (FIG. 3D).

### Example 4: Higher Level of Adult Hemoglobin with Enhanced Oxygen Carrying Ability

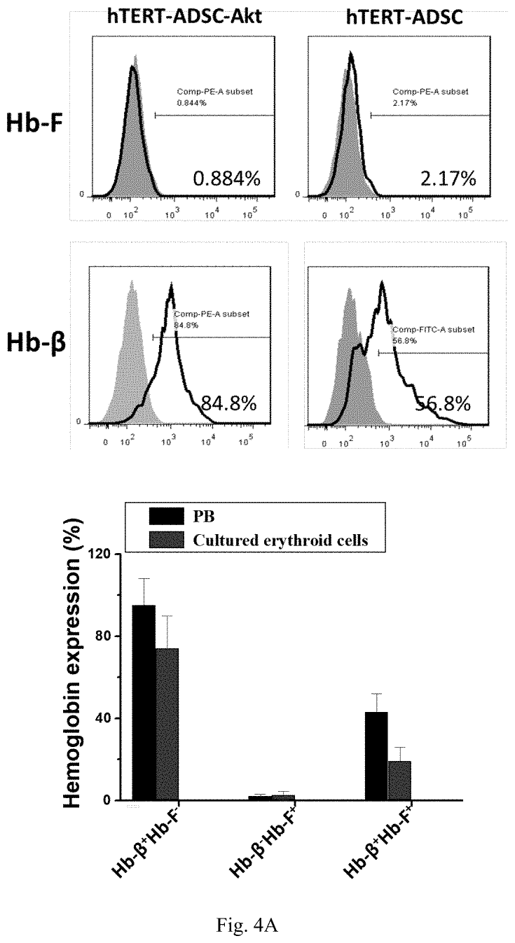

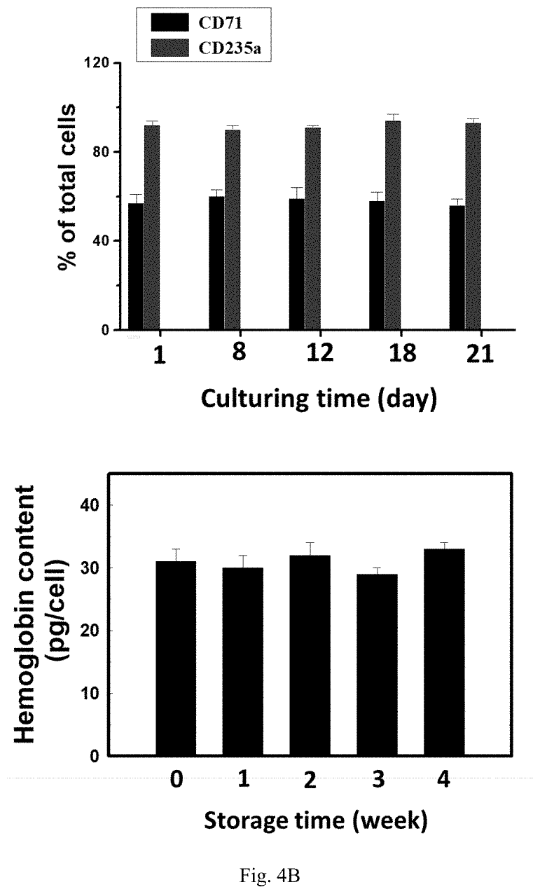

To examine hemoglobin subtypes by flow cytometry, although CB CD34+ cells mainly expressed both Fetal hemoglobin (Hb-F) and adult hemoglobin (Hb-β), cultured RBC mainly expressed more Hb-β up to 84.3±5.2% at day 21 in the hTERT-ADSC-Akt group than the hTERT-ADSC, which is comparable with normal adult peripheral blood (PB), respectively (FIG. 4A). Very few Hb-F positive cells were found and the mean proportion of Hb-β⁺Hb-F⁻ increased from day 21 (FIG. 4A).

For long-term storage of cultured RBCs, they were collected on day 28 and conserved at 4° C. in a preservative solution (CPDA-1) for 4 weeks. The erythroid markers and hemoglobin content remained unchanged during storage (FIG. 4B).

### Example 5: Maturation of Cultured Red Blood Cells (cRBC) in the NOD/SCID Model

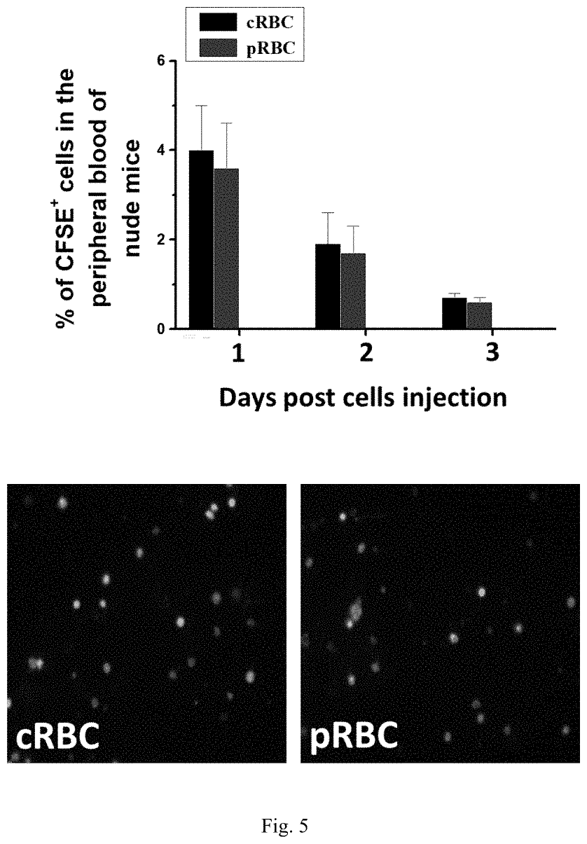

To investigate whether cultured red blood cells (cRBC) will mature in vivo, we injected CFSE-labeled adult peripheral blood RBC (pRBC) or cRBC collected on days 21-23 into CL2MDP-liposome-treated NOD/SCID or nude mice. For 3 days post-injection, CFSE⁺ cells were detected in the peripheral blood of mice in both groups of RBC (FIG. 5). At 3 days after injection, the percentage of CFSE⁺ cRBC decreased gradually and maintained in the mice circulation to the same extent as CFSE⁺ pRBC by confocal microscopy.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

What is claimed is:

1. A method for producing erythroid cells and/or erythrocytes comprising culturing hematopoietic stem cells (HSCs) or erythroid cells with a population of immortalized mesenchymal stem cells (MSCs) or conditioned medium obtained from the immortalized MSCs, wherein the immortalized MSCs are genetically engineered with a survival gene.

2. The method of claim 1, wherein the HSCs are CD34⁺ HSCs.

3. The method of claim 1, wherein the HSCs are derived from human umbilical cord blood.

4. The method of claim 1, wherein the survival gene is Akt gene.

5. The method of claim 1, wherein the immortalized MSCs are immortalized with human telomerase reverse transcriptase (hTERT).

6. The method of claim 1, wherein the MSCs are umbilical cord mesenchymal stem cells (UMSCs), adipose derived mesenchymal stem cells (ADSCs), or bone marrow mesenchymal stem cells (BMSCs).

7. The method of claim 1, wherein the immortalized MSCs are CD146⁺IGF1R⁺.

8. The method of claim 1, wherein the immortalized MSCs are hypoxia treated.

9. The method of claim 1, wherein the cell counts of the HSCs or erythroid cells to the cell counts of the immortalized MSCs range from about 100:1 to about 1:100.

10. The method of claim 1, which comprises enhancing HSC proliferation by culturing the HSCs with the immortalized MSCs or conditioned medium obtained from the immortalized MSCs.

11. The method of claim 10, which further comprises culturing the HSCs with at least one of stem cell factor (SCF), fms like tyrosine kinase 3 (Flt-3), interleukin 3 (IL-3), vitamin C, and dexamethasone.

12. The method of claim 1, which comprises inducing the HSCs to differentiate into the erythroid cells by culturing the HSCs with the immortalized MSCs or conditioned medium obtained from the immortalized MSCs.

13. The method of claim 12, which further comprises culturing the HSCs with at least one of SCF, erythropoietin (EPO), granulocyte-macrophage colony-stimulating factor (GM-CSF), Flt-3, dexamethasone, IL-3, vitamin C, and platelet rich plasma (PRP).

14. The method of claim 1, which comprises promoting differentiation and maturation of the erythroid cells by culturing the erythroid cells with the immortalized MSCs or conditioned medium obtained from the immortalized MSCs.

15. The method of claim 12, which further comprises culturing the erythroid cells with at least one of heparin, transferrin, SCF, EPO, and vitamin C.

16. The method of claim 1, which comprises enhancing HSC proliferation by culturing the HSCs with the immortalized MSCs or conditioned medium obtained from the immortalized MSCs; inducing the HSCs to differentiate into the erythroid cells comprising culturing the HSCs with the immortalized MSCs or conditioned medium obtained from the immortalized MSCs; and promoting differentiation and maturation of the erythroid cells by culturing the erythroid cells with the immortalized MSCs or conditioned medium obtained from the immortalized MSCs.

17. A method of making a blood product for use in transfusions comprising the method of claim 1.

18. A method for increasing hemoglobin synthesis comprising the method of claim 1.

19. The method of claim 18, wherein the hemoglobin is adult hemoglobin.
